(19) United States
(12) Patent Application Publication
(10) Pub. No.: US 2026/0258207 A1
(43) Pub. Date: Sep. 3, 2026

(54) METHOD AND DEVICE FOR PRODUCING A MICROFIBRILLATED CEULLOSE FILM

(71) Applicant: Stora Enso OYJ, Helsinki (FI)

(72) Inventors: Otto Nylén, Helsinki (FI); Riku Pihko, Jyväskylä (FI); Jarmo Kouko, Jyväskylä (FI); Oleg Timofeev, Jyväskylä (FI); Timo Rantanen, Jyväskylä (FI)

(73) Assignee: Stora Enso OYJ, Helsinki (FI)

(21) Appl. No.: 18/872,955

(22) PCT Filed: Jun. 9, 2023
(86) PCT No.: PCT/IB2023/055963

§ 371 Date: Dec. 9, 2024

(30) Foreign Application Priority Data
Jun. 10, 2022 (SE) 2230188-1

(51) Int. Cl. C08J 5/18

(52) U.S. Cl. CPC C08J 5/18; C08J 2301/02

(57) ABSTRACT

A method of producing a microfibrillated cellulose, MFC, film from an MFC dispersion, comprises subjecting a wet MFC film on a support to at least one drying step to form a dry MFC film (F′), measuring at least one parameter indicative of the dry solids content in the wet MFC film (F) and/or in the dry MFC film (F′) at at least two data points which are laterally spaced across at least a width of the MFC film (F, F′), and varying drying conditions of at least one of said at least one drying step across a width of the wet MFC film (F, F′) in response to said parameter. There is also disclosed a device for producing MFC film, use of such device and an MFC film.

### TECHNICAL FIELD

The present disclosure relates to a method and a device for producing microfibrillated cellulose, MFC, films. The disclosure relates particularly to a method which may provide a high quality MFC film. The disclosure further relates to devices for producing such MFC films and to the use of such devices for producing an MFC film. The disclosure also relates to an MFC film, which is produced according to the method.

### BACKGROUND

Microfibrillated cellulose (“MFC”) shall in the context of the patent application mean a cellulose particle, fiber or fibril having a width or diameter of from 20 nm to 1000 nm.

Various methods exist to make MFC, such as single or multiple pass refining, pre-hydrolysis followed by refining or high shear disintegration or liberation of fibrils. One or several pre-treatment steps is usually required in order to make MFC manufacturing both energy efficient and sustainable. The cellulose fibers of the pulp used when producing MFC may thus be native or pre-treated enzymatically or chemically, for example to reduce the quantity of hemicellulose or lignin. The cellulose fibers may be chemically modified before fibrillation, wherein the cellulose molecules contain functional groups other (or more) than found in the original cellulose. Such groups include, among others, carboxymethyl (CM), aldehyde and/or carboxyl groups (cellulose obtained by N-oxyl mediated oxidation, for example “TEMPO”), or quaternary ammonium (cationic cellulose). After being modified or oxidized in one of the above-described methods, it is easier to disintegrate the fibers into MFC.

MFC can be produced from wood cellulose fibers, both from hardwood and/or softwood fibers. It can also be made from microbial sources, agricultural fibers such as wheat straw pulp, bamboo, bagasse, or other non-wood fiber sources. It can be made from pulp, including pulp from virgin fiber, e.g. mechanical, chemical and/or thermomechanical pulps. It can also be made from broke or recycled paper or other packaging material.

Current research indicates that MFC may be a suitable material for packaging and coating of packaging, due to its barrier properties. Hence, MFC has the potential of replacing or supplementing currently used barrier films and layers, including polymer and metal films and coatings.

Forming of MFC films can be achieved by solvent casting of a viscous or gel-like fluid material on a support, such as a continuous conveyor belt, followed by dewatering/drying (e.g. evaporation) of the solvent.

The term “solvent casting” is a known term designating methods wherein a film is produced by applying a wet film comprising a film forming component which is distributed in a medium that is to be essentially removed, for example by dewatering and/or evaporation. The film forming component may be dispersed in a dispersing medium or dissolved in a solvent, hence the term “solvent casting”.

In the following, the term “MFC dispersion” will be used as reference to a dispersion/suspension or solution containing MFC and a dispersing medium, which is frequently water. The MFC dispersion will be in a viscous state.

Utilization of MFC films in packaging applications involves a specific challenge due to brittleness of the MFC film. Elasticity and ductility of the MFC film are associated with the water present in the MFC film, and the brittleness occurs when the MFC film is too dry. The dryness and subsequent brittleness can be a local effect in the MFC film, especially at the edge area of the film. In a casting process the lateral edges of the MFC film tend to dry faster than the middle parts of the film, which results in cross-machine (CD) directional differences in water content and ductility and subsequently possibly brittle edges. Due to brittleness, especially at the edge areas, MFC films may tend to break easily in converting processes. The problem is not necessarily limited to narrow and low speed appearing in lab and pilot-scale fabrication processes and use of films, but becomes more critical when operating film manufacturing on wider machines and/or in bigger scale and in higher production speeds.

A known approach is to use plasticizers and humectants, such as sugar alcohols (e.g. sorbitol) or polyethylene glycol, to make MFC films more ductile. Such hygroscopic additives and the water that they bind to the film may, however, interfere with bonding of the microfibrils, increase the strain-at-break value and decrease the tensile strength. Use of such additives improves the overall level of film ductility, but sometimes the films still are too brittle and the problem with cross-machine directional variation of water content of the film, and brittle edges, may also remain.

An additional problem relates to manufacturing of MFC films with casting technology when impingement drying, infra-red (IR) drying, or any other thermal drying from top of the film is used to dry films on a non-porous casting support. The casting support needs to be slightly wider in cross-machine direction than the wet MFC film deposited on it. However, since the heat flux applied from above increases the temperature of the casting support edges which have no wet MFC layer on them, the lateral edge portions of the film will be subjected to additional heat. This additional heat at the casting support edges makes the MFC film dry faster at the edges. MFC films are very thin and get easily overdried.

Moreover, when MFC films are allowed to have an uneven moisture profile in CD, the release behavior or adhesion of dry or semi wet MFC films to the casting support changes and becomes unpredictable or has higher variations. Since the film dries faster at the edges than at the middle, the edges tend to lose the adhesion to the casting support earlier. This also results in deformed and damaged edges in the film and web breaks in MFC film manufacturing.

Hence, there is a need for improvements in the drying of a wet MFC film on a support, as well as in MFC film properties.

### SUMMARY

It is an object to provide a method and a system, which provide improved MFC film quality, preferably with limited or no increase in production cost and more preferably with a reduction in production cost. A particular object is to address problems associated with inhomogeneous film properties, such as barrier properties, local brittleness, and/or problems with runnability in film manufacturing and converting.

The invention is defined by the appended independent claims, with embodiments being set forth in the appended dependent claims, in the following description and in the attached drawings.

According to a first aspect, there is provided a method of producing a microfibrillated cellulose, MFC, film from an MFC dispersion. The method comprises providing an MFC dispersion comprising a dispersing medium and a film forming component comprising about 50-100% by weight MFC, applying a layer of the MFC dispersion to a support to form a wet MFC film, subjecting the wet MFC film on the support to at least one drying step to form a dry MFC film, measuring at least one parameter indicative of the dry solids content in the wet MFC film and/or in the dry MFC film at at least two data points which are laterally spaced across at least a width of the MFC film, and varying drying conditions of at least one of said at least one drying step across the width of the wet MFC film in response to said parameter.

A content of the dispersing medium of the MFC dispersion may be at least 75% by weight, preferably more than 80% by weight, more than 85% by weight, more than 90% by weight or more than 95% by weight. The film forming component may comprise, consist of or consist essentially of MFC, optionally with one or more water soluble polymers which may operate as co-additives and/or co-film formers. Thus, the MFC dispersion comprises a dispersing medium and a film forming component, wherein the film forming component comprises 50-100% by weight MFC (i.e. based on the total dry weight of the film forming component). For example, the film forming component may comprise, in addition to MFC, a water soluble polymer that can form a film and/or improve bonding between the cellulose fibrils. Typical examples of such polymers are natural gums or polysaccharides or derivatives thereof such as e.g. carboxymethylated cellulose (CMC), starch, or polyvinyl alcohol (PVOH) or analogues thereof. The film forming component may also comprise further additives, such as one or more property-modifying additives and/or fillers. Non-limiting examples of such additives/chemicals are softeners and plasticizers, such as glycols, sugar alcohols such as sorbitol or polysaccharides such as sorbitol or glucose, film forming agents such as PVOH, carboxymethylated cellulose or methylcellulose, fillers, pigments, retention chemicals and dispersants or other polyelectrolytes, latexes, cross-linkers, optical dyes, fluorescent whitening agents, de-foaming chemicals, salts, pH adjustment chemicals, surfactants, biocides and/or optical chemicals. The film forming component may also comprise other natural fibre material in addition to MFC.

The dispersing medium may comprise water and optionally one or more solvents.

The layer of the MFC dispersion may be applied to the substrate by a casting technique.

The support may be a non-porous support and in particular a continuous non-porous support, such as a metal belt, in particular a steel belt, a polymer belt or a polymer coated belt. A metal belt may be coated, e.g., with ceramic material.

A parameter indicative of dry solids content may, as non-limiting examples, be a temperature, as temperature typically rises as the dry solids content increases, or a material composition that can be determined by various spectroscopic methods.

The data points may be spaced in at least the cross machine direction (CD), which is typically a direction that is perpendicular to a direction in which the MFC film travels during drying, i.e. a machine direction (MD). For example, the data points may be spaced only in the CD or they may be spaced both in the CD and in the MD.

By varying the drying conditions based on the measured dry solids content, i.e. the measured at least one parameter indicative of the dry solids content, of the MFC dispersion or MFC film during its production, it is possible to ensure or at least promote that the film dries substantially evenly, and in particular that the film does not become too dry at edge portions. Hence, distribution of moisture, and of some additives, in the dry MFC film can be improved, thus reducing problems with local brittleness, etc. The method defined above allows for the dispersing medium content of the dry MFC film to be controlled to a desired value with increased precision, which facilitates the handling of the resulting dry film, such as peeling it off the support, and subsequent packaging and handling.

In addition, the content of dispersing medium and of additives, which follow the dispersing medium, may have an impact on e.g. barrier properties of the finished film. For example, areas with lower dry solids content, which may arise due to locally faster drying, can be identified as areas wherein soluble additives may be present in a higher concentration.

The dry film may be considered as a thin continuous sheet formed material. Depending on its composition, purpose and properties, the dry film may also be considered as a thin paper or web, or even as a membrane.

In practical embodiments, a large number of data points may be provided, to thus allow for a detailed profile of the dry solids content of the film to be derived.

The measuring step may be performed after at least part of said at least one drying step.

Hence, the measuring may be performed after one or more drying steps, such as after the entire drying process, or measuring may be performed in between drying steps.

The method may further comprise at least one dewatering step prior to said at least one drying step, wherein said measuring step may be performed after at least part of said at least one dewatering step and before said at least one drying step.

Hence, the measuring may be performed after one or more dewatering steps, such as after the entire dewatering step, but before a drying step, or in between sub-steps in the dewatering process. Alternatively or additionally, the at least one drying step may form a pre-drying step, which is operated prior to the dewatering step. As yet another option, measuring may take place after the dry MFC film has been separated from the support. In such cases, measurements may be made on the top side and/or on the back side of the dry MFC film.

The method may further comprise a pre-drying step, which is performed prior to the dewatering step and wherein said measuring step is performed after the pre-drying step and before the dewatering step.

At least one of the data points may indicate dry solids content at a position in a lateral edge portion of the MFC film.

The lateral edge portion may be defined as an area which extends in a direction perpendicular to a longitudinal direction (which is parallel with a production direction, MD) of the MFC film by a distance of 50 mm, preferably 30 mm or 10 mm, from an outermost edge (lateral edge) of the MFC film.

At least one of the data points may indicate dry solids content at a portion that is laterally spaced from a lateral edge portion of the MFC film.

The concept of varying drying conditions may comprise selectively varying a drying effect across the width direction.

For example, said varying a drying effect may comprise reducing the drying effect at at least one lateral edge portion of the wet MFC film.

In particular, said varying a drying effect may comprise limiting a drying width to be narrower than an MFC film width.

For example, said varying a drying effect may comprise limiting an impingement width of a drying gas and/or radiation (microwave, infra-red (IR)) to be narrower than an MFC film width.

The drying gas may be air or any other type of gas that is suitable for the purpose.

As another example, said varying a drying effect may comprise guiding drying gas away from at least one lateral edge portion of the MFC film,

As another example, said varying a drying effect may comprise extracting drying gas away from at least one lateral edge portion of the MFC film.

As another example, said varying a drying effect may comprise masking a lateral edge portion of the MFC film from e.g. drying gas and/or radiation.

As another example, said varying a drying effect may comprise sealing against a support surface laterally outside a lateral edge portion of the MFC film so as to prevent drying gas and/or radiation from reaching the support.

Hence the heating of the support can be reduced, leading to a reduction in the drying effect at edge portions of the film.

It is further possible to vary the drying effect by controlling a temperature profile of the support, e.g. by controlling heating and/or cooling of the support from below.

As another example, said varying a drying effect may comprise selectively controlling a duty cycle, a combustion gas pressure and/or an intensity of a radiation source for providing said drying.

As another example, said varying drying conditions may comprise selectively injecting at least one of a cooling medium and a dispersing medium onto the wet MFC film.

As a non-limiting example, water may be used as cooling and/or dispersing medium.

As another example, said varying drying conditions may comprise cooling at least one support edge portion laterally outside the wet MFC film by applying the cooling medium.

Said varying drying conditions may comprise applying said cooling medium on a lateral edge portion of the MFC film and/or applying the dispersing medium on a lateral edge portion of the MFC film.

The method may further comprise separating the dry MFC film from the support and winding the separated MFC film onto a reel.

Hence, the MFC film will remain on the support throughout the entire drying (and dewatering, if any) process.

In the method, the provided MFC dispersion may have a dry solids content of about 2.5-25% by weight, preferably about 2.5-15% by weight or 2.5-10% by weight or about 2.5-8% by weight.

Regardless of the dry solids content, a viscosity of the MFC dispersion may be above about 4 Pas at a shear rate of 20 s⁻¹. The viscosity may be determined for a dispersion at a temperature of about 20-80 deg C and preferably about 20-60 deg C. A preferred method of measuring viscosity is by use of a rheometer using bob-and-cup geometry, such as an Anton Paar MCR 302 dynamic rotational rheometer.

In the method, an average film thickness of the dry MFC film may be about 5-60 μm, preferably 10-50 μm, 15-45 μm or 20-40 μm. The average film thickness may be defined as an average thickness of the film across the entire width.

In the method, a film weight of the dry MFC film may be about 4-80 g/m², preferably 8-67 g/m², 12-60 g/m², 16-53 g/m² or 20-45 g/m².

In the method, a dispersing medium content of the dry MFC film may be about 0.1-20% by weight, preferably 1-15% by weight, or 2-14% by weight.

In the method, a film forming component content of the dry MFC film (F′) may be at least 80-99.9% by weight, preferably 85-99% by weight or 86-98% by weight.

In the method, the film forming component may comprise at least 60% by weight MFC, preferably at least 70% by weight MFC or at least 80% by weight MFC.

In the method, a film width of the dry MFC film may be about 0.3-4 m, preferably 0.5-4 m, 1-4 m or 2-4 m.

The method may further comprise measuring said at least one parameter indicative of the dry solids content in the wet MFC film and/or in the dry MFC film at at least two data points which are spaced from each other in a thickness direction of the MFC film.

Measurement data indicating the dry solids content at various points which are spaced apart in the thickness direction can be stored and used for following up MFC film quality and/or for grading the resulting MFC film.

It is also possible to use such data for assessing the drying performance and thus as a basis for the step of varying drying conditions across the width of the wet MFC film.

According to a second aspect, there is provided a device for producing a microfibrillated cellulose, MFC, film from an MFC dispersion. The device comprises a support guiding device, configured to guide a continuous support, a casting device, configured to apply the MFC dispersion as a wet MFC film on the support, a drying arrangement, configured for removing a dispersing medium from the wet MFC film so as to form a dry MFC film, at least one measuring arrangement, configured for providing data indicative of a dry solids content in the wet MFC film and/or in the dry MFC film at at least two data points which are laterally spaced across at least a width of the MFC film, and a controller, configured to receive said data, and to control the drying arrangement based on said data, so as to vary drying conditions across a width of the wet MFC film.

The device may be configured for carrying out the method or methods as disclosed above.

The support guide may be configured to guide a support having a width of at least about 0.3-4 m, preferably 0.5-4 m, 1-4 m or 2-4 m.

The support may be a non-porous support and in particular a continuous non-porous support, such as a metal belt, in particular a steel belt, a polymer belt or a polymer coated belt. A metal belt may be coated, e.g., with ceramic material.

The device may further comprise a dewatering arrangement upstream said drying arrangement, wherein said measuring arrangement is arranged downstream of at least part of the dewatering arrangement and upstream of the drying arrangement.

Hence, the measuring may be performed after one or more dewatering steps, such as after the entire dewatering step, but before a drying step, or in between sub-steps in the dewatering process.

It is understood that the measuring arrangement may comprise a first measuring arrangement arranged at least partially downstream of the drying arrangement and a second measuring arrangement arranged at least partially downstream of the dewatering arrangement.

The device may further comprise a pre-drying arrangement, upstream said dewatering arrangement, wherein said measuring arrangement may be arranged downstream of at least part of the pre-drying arrangement and upstream of the dewatering arrangement.

A further measuring arrangement, or part of the measuring arrangement, may thus be arranged downstream of at least part of the pre-drying arrangement.

According to a third aspect, there is provided use of a device as described above for forming a dry MFC film having a dispersing medium content of about 0.1-20% by weight, preferably 1-15% by weight, or 2-14% by weight, from an MFC dispersion having a dry solids content of about 2.5-25% by weight, preferably 2.5-15% by weight or 2.5-10% by weight or about 2.5-8% by weight, and a viscosity which is above about 4 Pas at a shear rate of 20 s⁻¹.

According to a fourth aspect, there is provided a microfibrillated cellulose, MFC, film having a longitudinal direction, which is parallel with a production direction of the film and a width direction, which is perpendicular to the longitudinal direction, the MFC film having a film forming component content of at least 80-99.9% by weight, preferably 85-99% by weight or 86-98% by weight, the film forming component comprises at least 50% by weight MFC, the MFC film having a width of about 0.3-4 m, preferably 0.5-4 m, 1-4 m or 2-4 m, the MFC film having a dispersing medium content of about 0.1-20% by weight, preferably 1-15% by weight, or 2-14% by weight, and the dispersing medium content of the MFC film having a standard deviation which is less than 1% by weight along the width direction, where the moisture content is analyzed for each cm of MFC film width.

In the MFC film, the film forming component may comprise at least 60% by weight MFC, preferably at least 70% by weight MFC or at least 80% by weight MFC.

An average film thickness may be about 5-60 μm, preferably about 10-50 μm, about 15-45 μm or about 20-40 μm.

A film weight may be about 4-80 g/m², preferably about 8-67 g/m², about 12-60 g/m², about 16-53 g/m² or about 20-45 g/m².

### DRAWINGS

FIGS. 1a-1b schematically illustrate a film forming device.

FIG. 2 is a schematic sectional view taken along the line A-A in FIG. 1a, illustrating a first embodiment of a drying arrangement.

FIGS. 3a-3c are schematic sectional views taken along the line A-A in FIG. 1a, illustrating different versions of a second embodiment of a drying arrangement.

FIG. 4 is a schematic sectional view taken along the line A-A in FIG. 1a, illustrating a third embodiment of a drying arrangement.

FIG. 5 is a schematic sectional view taken along the line A-A in FIG. 1a, illustrating a fourth embodiment of a drying arrangement.

FIG. 6 is a schematic sectional view taken along the line A-A in FIG. 1a, illustrating a fifth embodiment of a drying arrangement.

FIG. 7 is a schematic sectional view taken along the line A-A in FIG. 1a, illustrating a sixth embodiment of a drying arrangement.

FIG. 8 is a schematic sectional view taken along the line A-A in FIG. 1a, illustrating a seventh embodiment of a drying arrangement.

FIG. 9 is a schematic sectional view taken along the line B-B in FIG. 1a, illustrating a first embodiment of a measuring arrangement.

FIG. 10 is a schematic sectional view taken along the line B-B in FIG. 1a, illustrating a second embodiment of a measuring arrangement.

FIG. 11 is a schematic sectional view taken along the line B-B in FIG. 1a, illustrating a third embodiment of a measuring arrangement.

### DETAILED DESCRIPTION

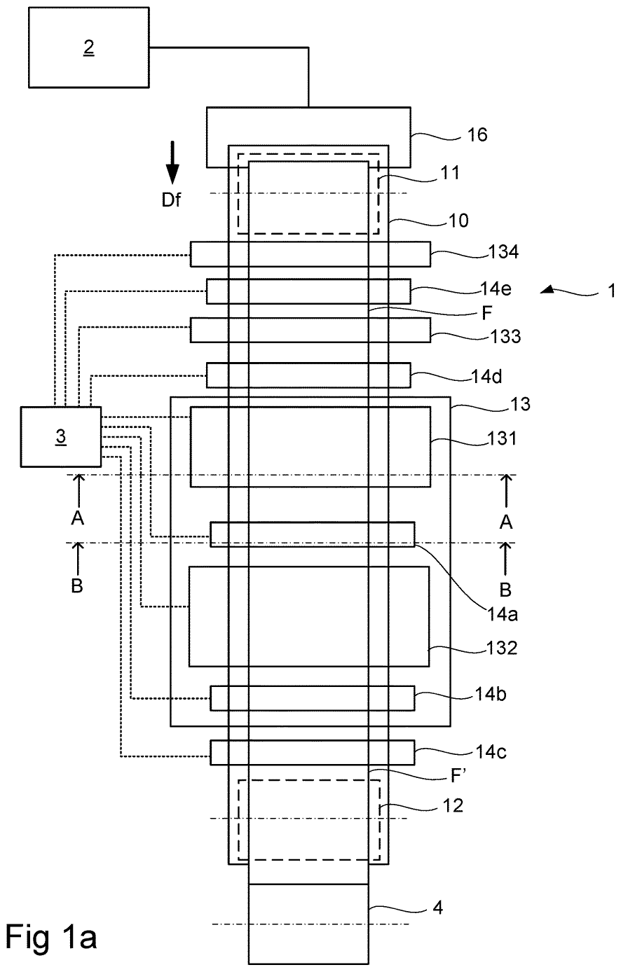

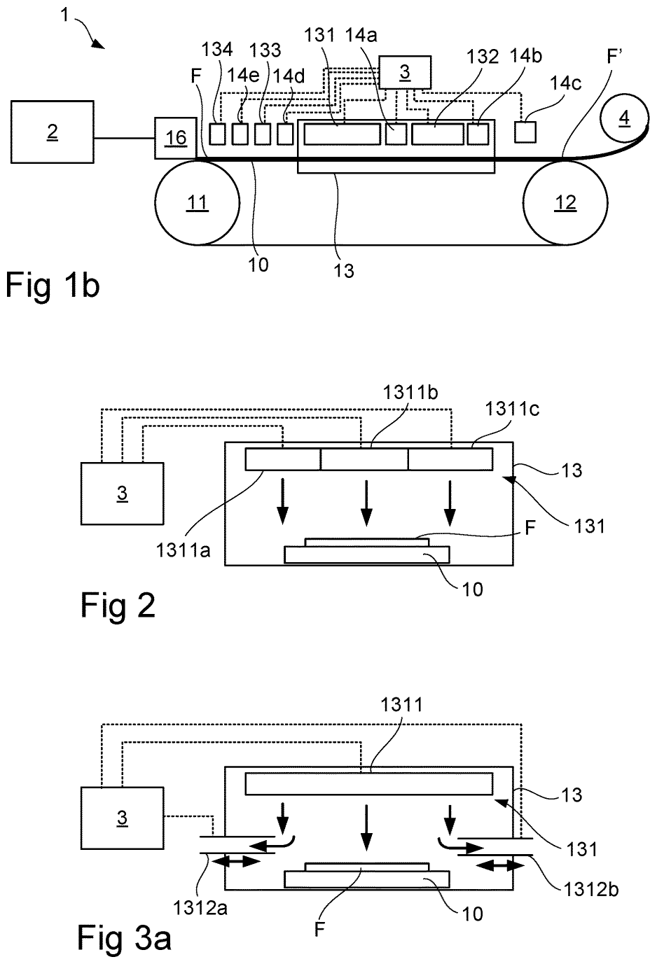

Referring to FIG. 1a, there is schematically illustrated a top view of a film forming device 1. FIG. 1b schematically illustrates a side view of the film forming device 1.

In the present disclosure, the film forming device 1 will be illustrated with reference to a film forming device 1 for forming a non-laminated film, or “free-standing film”, i.e. a film that is not laminated to any substrate material. Hence, as illustrated in FIGS. 1a-1b, the finished film F′ is stripped off a support 10 and rolled onto a reel 4.

The support 10, from which the dry film F′ is to be stripped off may be provided in the form of a non-porous and preferably endless belt, such as a metal belt, e.g. a steel belt or a polymer, or polymer coated, belt. A metal belt may be coated by e.g. a ceramic coating. The support 10 is preferably non-porous, so as to provide for a smooth film surface. In particular, the support may be polished to mirror gloss. In the illustrated embodiment, the support 10 is an endless support which runs over a support guide in the form of a pair of pulleys 11, 12.

An MFC supply 2 is provided for supplying an MFC dispersion to a casting device 16, which is configured to deposit the MFC dispersion as a thin, wet film F having an even thickness. The support 10 carrying the wet film F is passed through a dryer 13, which may comprise one or more drying arrangements 131, 132. In the case with more than one drying arrangement 131, 132, the drying arrangements may be identical with each other, or they may differ in terms of e.g. length. Also, the drying arrangements 131, 132 may be individually controlled, such that they provide different drying parameters.

Optionally, a dewatering arrangement 133, such as a dewatering device, which may include a press, may be provided upstream of the drying arrangements 131, 132. Such dewatering arrangements 133 are known as such.

For example, the dewatering may be performed by applying a press fabric in direct contact with the wet MFC film and conducting the wet MFC film, arranged between the press fabric and the support, through a pressing equipment. Alternatively, the dewatering may be performed by applying a porous wire or membrane in direct contact with the wet MFC film and conducting the wet MFC film, arranged between the porous wire or membrane and the support, through a vacuum dewatering equipment, in which the porous wire or membrane is covering one or several vacuum cavities that causes dispersing medium to be removed from the wet MFC film.

Further, optionally, a pre-drying arrangement 134 may be provided upstream of the dewatering arrangement 133.

In other embodiments, the drying arrangements 131, 132, and pre-drying arrangement 134, if any, may use the same or different drying techniques, e.g. each one being selected according to the non-limiting options mentioned herein.

One or more measuring arrangements 14a, 14b, 14c, 14d, 14e for measuring at least one parameter indicative of a dry solids content in the wet MFC film F and/or in the dry MFC film F′ are provided, either inside the dryer 13 or outside the dryer 13. For example, a measuring arrangement 14a may be provided inside the dryer 13, between a pair of drying arrangements 131, 132. As another example, a measuring arrangement 14b may be provided inside the dryer 13 and downstream of the drying arrangements 131, 132. As yet another example, a measuring arrangement 14c may be provided downstream of and outside the dryer 13. For example, a cooling arrangement (not shown) may be provided between the dryer 13 and the measuring arrangement 14c.

It is understood that one, two, three, four or all of the above illustrated measuring arrangements 14a, 14b, 14c, 14d, 14e may be provided.

Where a dewatering arrangement 133 is provided upstream of the drying arrangement 131, 132, a measuring device 14d may be provided downstream of the dewatering arrangement 133 and upstream of the drying arrangement 131, 132. In some embodiments, the measuring arrangement, or a sub-part thereof, may be provided downstream of part of the dewatering arrangement 133, such as between sub-steps of the dewatering arrangement 133, or between dewatering arrangements, if several dewatering arrangements are provided.

It is also possible to provide a measuring arrangement 14e upstream of the dewatering arrangement 133.

A controller 3 may be provided for controlling at least the dryer 13 and the measuring arrangements 14a, 14b, 14c, 14d, 14e. Optionally, the controller may control further, or all, functions of the film forming device 1. The controller may also control the dewatering arrangement 133, if any, as well as the pre-drying arrangement 134, if any.

FIG. 2 is a schematic sectional view taken along the line A-A in FIG. 1a, illustrating a first embodiment of a drying arrangement, which may be arranged in a dryer 13.

Drying and pre-drying may be performed by evaporation, impingement drying with hot gas or hot air, infra-red (IR) drying, microwaves, near infrared drying, UV drying, radiation drying, thermal heating, heating the support with steam or electricity or any other method or combination of methods well known in the art.

It is understood that any of the herein mentioned drying techniques can be made controllable across the width of the support 10, and optionally also along the length of the film. For example, also techniques that heat the support 10 from below can easily be divided into zones, as desired.

The drying arrangement illustrated in FIG. 2 is a controllable convection drying arrangement, for selectively feeding a dry and preferably also warm or hot gas, such as air, towards the wet film F.

In the drying arrangement illustrated in FIG. 2, three individually controllable convection zones 1311a, 1311b, 1311c are provided. It is understood that the number of convention zones 1311a, 1311b, 1311c can be selected depending on the width of the film and support and on what resolution is required.

For each convection zone 1311a, 1311b, 1311c, a gas flow rate and/or a gas flow direction and/or a gas temperature and/or a gas composition (e.g. moisture content) may be individually controllable by the controller 3, either by controlling a respective blower, a respective nozzle, a respective heater and/or a respective gas mixer. Alternatively, or as a supplement, the supply of the gas to the respective convection zone 1311a, 1311b, 1311c may be controlled by a valve (not shown).

Hence, during operation, the drying effect in each of the zones 1311a, 1311b, 1311c is individually controlled by the controller 3. For example, it is possible to provide a lower gas flow and/or lower temperature and/or higher moisture content at the zones 1311a, 1311c near the lateral edges of the film F, so as to reduce the drying effect at the lateral edge portions of the film F.

In the embodiment of FIG. 2, it is possible to provide for a drying gas impingement width to be narrower than that of the MFC film F, F′. For example, the impingement width may be 30-70 mm, preferably 40-60 mm or about 50 mm, narrower than an MFC film F, F′ width.

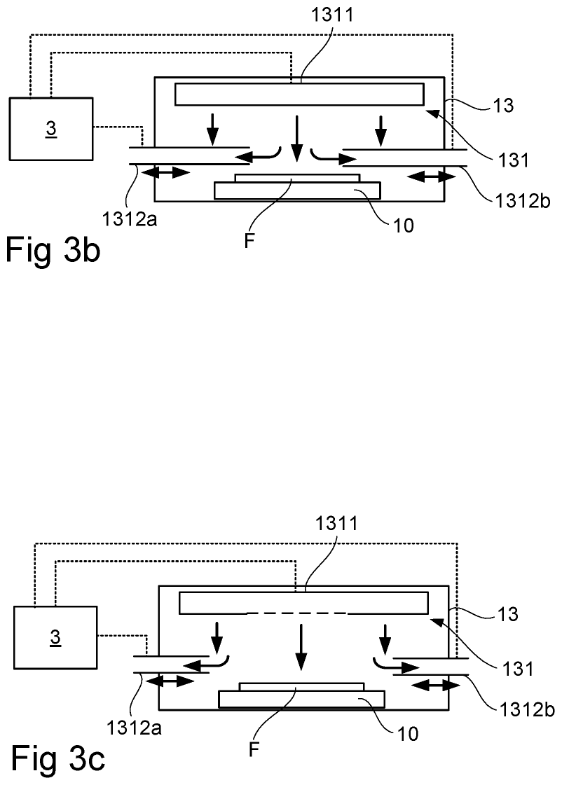

FIG. 3a is a schematic sectional view taken along the line A-A in FIG. 1a, illustrating a first version of a second embodiment of a drying arrangement, which may be arranged inside a dryer 13.

In the drying arrangement illustrated in FIG. 3a, there is a single convection zone 1311, which may be operated in the same manner as one of the convection zones described with reference to FIG. 2, in that a gas flow rate and/or a gas flow direction and/or a gas temperature and/or a gas composition (e.g. moisture content) may be controllable by the controller 3.

In FIG. 3a, there is provided at least one evacuation outlet 1312a, 1312b, which may be laterally and/or vertically displaceable, optionally controllable by the controller 3, such that its outlets can be positioned at a desired lateral position relative to the film F. The evacuation outlets 1312a, 1312b may be connected to an extraction device, such as a fan, which may be controllable by the controller 3, such that an extraction rate is controllable by the controller 3.

Hence, during operation, the drying effect at lateral edge portions of the film F may be reduced by dry and/or hot air being extracted from the area at the lateral edge portions and thus being prevented from interacting with the wet film F, so as to reduce the drying effect at the lateral edge portions of the film F.

FIG. 3b schematically illustrates a second version of the second embodiment of a drying arrangement, wherein inlets to the evacuation outlets 1312a, 1312b are positioned laterally inwardly of MFC film F, F′ edges, such that the drying gas is prevented from reaching the MFC film edges.

FIG. 3c schematically illustrates a third version of the second embodiment of a drying arrangement, wherein the convection zone 1311 is configured to provide a greater flow of drying gas at a laterally central portion thereof, e.g. by providing reduced gas flow resistance at the central portion of the convection zone 1311 as compared with at edge portions of the convection zone 1311. This embodiment may be combined with the embodiments of FIGS. 3a and/or 3b.

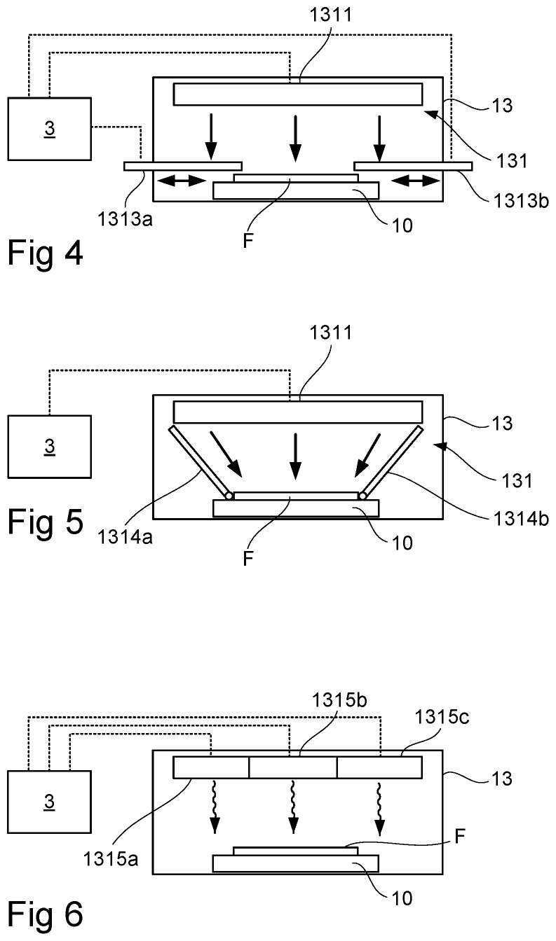

FIG. 4 is a schematic sectional view taken along the line A-A in FIG. 1a, illustrating a third embodiment of a drying arrangement, which may be arranged inside a dryer 13.

In the drying arrangement illustrated in FIG. 4, there is a single convection zone 1311, which may be operated in the same manner as the convection zone described with reference to FIGS. 3a-3c.

In FIG. 4, there is provided at least one shield 1313a, 1313b, which may be laterally displaceable, optionally controllable by the controller 3, such that the shields 1313a, 1313b can be positioned at a desired lateral position relative to the film F.

Hence, during operation, the drying effect at lateral edge portions of the film may be reduced as the incoming hot and/or dry gas is deflected from the lateral edge portions, so as to reduce the drying effect at the lateral edge portions of the film F.

FIG. 5 is a schematic sectional view taken along the line A-A in FIG. 1a, illustrating a fourth embodiment of a drying arrangement, which may be arranged inside a dryer 13.

In the drying arrangement illustrated in FIG. 5, there is a single convection zone 1311, which may be operated in the same manner as the convection zone described with reference to FIGS. 3a-3c.

In FIG. 5, there is provided at least one sealing arrangement 1314a, 1314b, which may seal against the support 10 immediately laterally outside the film F, such that the gas flow from the convection zone 1311 does not impinge on the support 10.

Hence, during operation, the hot gas is prevented from reaching the support 10, which may be a metal support and as such having higher heat coefficient than the film F, whereby the heating of the support laterally outside of the film F is reduced, and thus also the drying effect at the lateral edge portion of the film F.

FIG. 6 is a schematic sectional view taken along the line A-A in FIG. 1a, illustrating a fifth embodiment of a drying arrangement, which may be arranged inside a dryer 13.

The drying arrangement illustrated in FIG. 6 is a controllable radiation drying arrangement, for selectively projecting radiation, such as infra-red (IR) radiation towards the wet film F.

In the drying arrangement illustrated in FIG. 6, three individually controllable radiation zones 1315a, 1315b, 1315c are provided. It is understood that the number of radiation zones 1315a, 1315b, 1315c can be selected depending on the width of the film F and the support 10 and on what resolution is required.

For each radiation zone 1315a, 1315b, 1315c, a radiation intensity and/or a radiation duty cycle may be individually controllable, by the controller 3, e.g. by controlling the respective radiation source and/or by controlling a radiation filter or valve.

Hence, during operation, the drying effect in each of the radiation zones 1315a, 1315b, 1315c is individually controlled by the controller 3. For example, it is possible to provide radiation in the radiation zones 1315a, 1315c near the film lateral edges, so as to reduce the drying effect at the lateral edge portions of the film F.

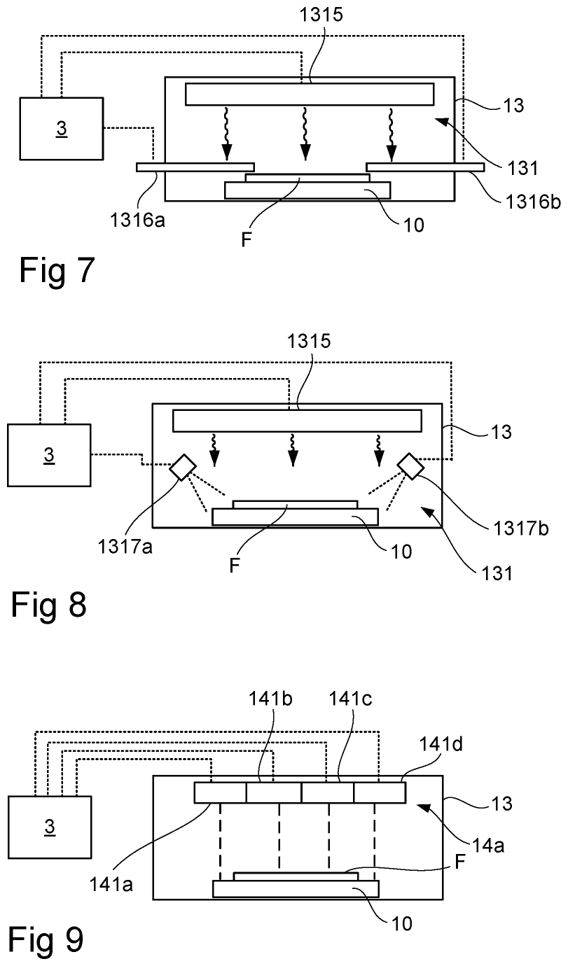

FIG. 7 is a schematic sectional view taken along the line A-A in FIG. 1a, illustrating a sixth embodiment of a drying arrangement, which may be arranged inside a dryer 13.

In the drying arrangement illustrated in FIG. 7, there is a single radiation zone 1315, which may be operated in the same manner as one of the radiation zones described with reference to FIG. 6.

In FIG. 7, there is provided at least one shield 1316a, 1316b, which may be laterally displaceable, optionally controllable by the controller 3, such that the shields 1316a, 1316b can be positioned at a desired lateral position relative to the film F. The shields may be completely non-transparent to the radiation. Alternatively, the shields may be variably, e.g. controllable by the controller 3, transparent to the radiation or partially transparent to the radiation.

The shields 1316a, 1316b may be operated so as to mask only the support 10 so as to reduce heating of the support 10, or so as to mask both the support 10 and lateral edge portions of the film F.

Hence, during operation, the drying effect at lateral edge portions of the film may be reduced as the incoming radiation is masked from the support 10 and optionally also from the lateral edge portions, so as to reduce the drying effect at the lateral edge portions of the film F.

FIG. 8 is a schematic sectional view taken along the line A-A in FIG. 1a, illustrating a seventh embodiment of a drying arrangement, which may be arranged inside a dryer 13.

In the drying arrangement illustrated in FIG. 8, there is a single radiation zone 1315, which may be operated in the same manner as one of the radiation zones described with reference to FIG. 6.

In FIG. 8, there is provided at least one injector 1317a, 1317b for a dispersing medium and/or a coolant agent. The injector 1317a, 1317b may be controllable by the controller 3 so as to selectively apply dispersing medium and/or coolant agent to the film F and/or to the support 10 just laterally outside the film, so as to increase the moisture level in the film, and/or to cool the film F and/or the support 10.

Hence, during operation, the drying effect at lateral edge portions of the film may be reduced as the moisture level of the film F is selectively increased and/or as the support 10 and optionally also the lateral edge portions are cooled, so as to reduce the drying effect at the lateral edge portions of the film F.

FIG. 9 is a schematic sectional view taken along the line B-B in FIG. 1a, illustrating a first embodiment of a measuring arrangement 14a, which may be arranged inside or outside a dryer 13.

In the measuring arrangement 14a illustrated in FIG. 9, a measuring sensor is connected to the controller 3 and may be formed as a 1D sensor (a line sensor) having a plurality of sensor zones 141a, 141b, 141c, 141d, each capable of generating sensor data for a laterally limited portion of the film F and optionally of the support 10. The number of sensor zones 141a, 141b, 141c, 141d may be arbitrarily selected depending on the required resolution.

Various sensing techniques may be utilized.

For example, the measuring sensor may be a temperature sensor, whereby each sensor zone 141a, 141b, 141c, 141d provides temperature data for a corresponding portion of the film F and optionally of the support 10, with a moisture level for each film portion being derivable based on the temperature for that film portion.

As further examples, the sensor may use infra-red (IR)spectroscopy or Raman spectroscopy to provide data corresponding to a composition of the film F, whereby a material composition may be derived based on resulting spectral data. Hence a 2D map of the dispersing medium content of the wet or dry MFC film F, F′ may be created.

It is possible to use spectroscopy methods, such as Raman spectroscopy or near infra-red (NIR) spectroscopy, to not only measure the dry solids content at a point on the surface of the wet or dry MFC film, but also to measure the dry solids content at various points along a thickness direction of the wet or dry MFC film F, F′. Hence, effectively, a 3D map of the dispersing medium content of the wet or dry MFC film F, F′ may be created.

The sensor may be operated continuously or at certain intervals to derive a temperature or composition profile of the film F, F′, which may be used as input to the controller 3 for determining how to operate the dryer 13.

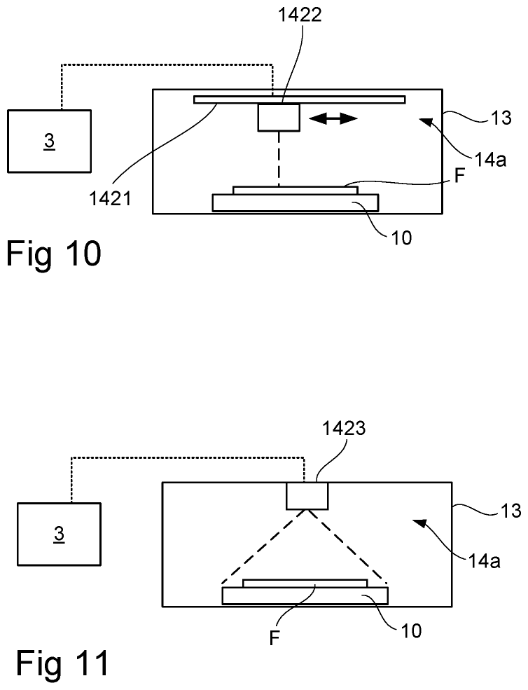

FIG. 10 is a schematic sectional view taken along the line B-B in FIG. 1a, illustrating a second embodiment of a measuring arrangement, which may be arranged inside or outside a dryer 13.

In the measuring arrangement illustrated in FIG. 10, a measuring sensor 1422 is connected to the controller 3 and may be formed as a point sensor, which may be scanned across the film F, F′ and optionally also across the support 10. Scanning may be achieved by displacing the measuring sensor along a guide 1421 and/or by using a beam guide.

The sensor may use any of the sensing techniques described with reference to FIG. 9.

As with the arrangement described with reference to FIG. 9, the sensor 1422 may be operated continuously or at certain intervals to derive a temperature or composition profile of the film F, F′, which may be used as input to the controller 3 for determining how to operate the dryer 13.

FIG. 11 is a schematic sectional view taken along the line B-B in FIG. 1a, illustrating a third embodiment of a measuring arrangement, which may be arranged inside or outside a dryer 13.

In the measuring arrangement illustrated in FIG. 11, a 2D sensor, such as a camera 1423, e.g. a hyperspectral camera or an infra-red (IR)camera, may be arranged such that a field of view covers the width of the film F, F′ and optionally also of the support 10.

The camera 1423 may thus use any of the sensing techniques described with reference to FIG. 9.

As with the arrangement described with reference to FIG. 9, the camera 1423 may be operated continuously or at certain intervals to derive a temperature or composition profile of the film F, which may be used as input to the controller 3 for determining how to operate the dryer 13.

At least one measuring arrangement as disclosed with respect to any one of FIGS. 9-11 may be applied at any position along the support 10 where a measuring arrangement 14a, 14b, 14c, 14d, 14e is being indicated.

A wet MFC film F can be formed from an MFC dispersion having a dry solids content about 2.5-4% by weight, about 4-6% by weight, about 6-8% by weight, about 8-10% by weight, about 10-12% by weight, about 12-14% by weight, about 14-16% by weight, about 16-18% by weight, about 18-20% by weight, about 20-22% by weight or about 22-25% by weight, which is considered a high dry solids content MFC. Preferably, the dry solids content may be greater than 3% or greater than 4% by weight.

Thickness of the dry film F′ may be measured using, as non-limiting examples, white light interferometry, laser profilometry, or optically by cutting a sample in cross-machine directional line (either cast in resin or not) and microscopic imaging (e.g. scanning electron microscopy or other applicable method) of the cut section in thickness direction.

The average dry film F′ thickness may be on the order of 5-60 μm, 15-20 μm, preferably 20-60 μm, 10-50 μm, 30-50 μm, 15-45 μm or 20-40 μm.

Particular dry film F′ thicknesses may be 5-10 μm, 10-15 μm, 15-20 μm, 20-25 μm, 25-30 μm, 30-35 μm, 35-40 μm, 40-45 μm, 45-50 μm, 50-55 μm or 55-60 μm.

A dry film F′ grammage may be on the order of 4-80 g/m², preferably 8-67 g/m², 12-60 g/m², 16-53 g/m² or 20-45 g/m².

Particular dry film F′ grammages may be 4-10 g/m², 10-20 g/m², 20-30 g/m², 30-40 g/m², 40-50 g/m², 50-60 g/m², 60-70 g/m² or 70-80 g/m².

A dispersing medium content of the dry film F′ may be on the order of 0.1-20% by weight, preferably 1-15% by weight, or 2-14% by weight.

Particular dispersing medium contents of the dry film F′ may be on the order of 0.1-1% by weight, 1-2% by weight, 2-3% by weight, 3-4% by weight, 4-5% by weight, 5-6% by weight, 6-7% by weight, 7-8% by weight, 8-9% by weight, 9-10% by weight, 10-11% by weight, 11-12% by weight, 12-13% by weight, 13-14% by weight or 14-15% by weight.

A film forming component content of the dry film F′ may be 80-99.9% by weight, preferably 85-99% by weight or 86-98% by weight, with the remainder being dispersing medium and/or one or more additives.

In particular, the film forming component may have an MFC content of 50-60% by weight, 60-70% by weight, 70-80% by weight, 80-90% by weight, 90-95% by weight or 95-99% by weight.

A width of the dry film F′ may be about 0.3-4 m, preferably 0.5-4 m, 1-4 m or 2-4 m.

Particular film F, F′ widths may be 0.3-0.5 m, 0.5-1 m, 1-1.5 m, 1.5-2 m, 2-2.5 m, 2.5-3 m, 3-3.5 m or 3.5-4 m. A corresponding support 10 width may be at least the same as the film width and in some cases about 10-20 cm wider than the film width.

By using the method of the present disclosure, it is possible to produce a dry MFC film F′ having an even composition with respect to the dispersing medium. By measuring the content of the dispersing medium at a plurality of points across the width of the dry MFC film F′, such as at 1 cm intervals across the entire width of the dry MFC film F′, it is possible to derive an average content of the dispersing medium, as well as a standard deviation of said content. With the method disclosed herein, it is possible to achieve a standard deviation of 1% by weight or less, also across the width of an MFC film F′ having a width which exceeds that of lab scale equipment.

It is also possible to impact the dry solids content of the film F, F′ by measures which may be taken at the casting device 16.

For example, it may be possible to adjust a nozzle of the casting device 16 so as to vary a thickness of the wet MFC dispersion layer that is applied to the support 10, for example so as to increase thickness in portions where the MFC film F, F′ dries faster.

It is also possible to vary the temperature at the nozzle, such that the temperature of the wet MFC film F, F′ applied to the support 10 will vary across the width of the support 10.

It is also possible to locally cool or heat the support 10 at or immediately downstream of the casting device 16.

1. A method of producing a microfibrillated cellulose, MFC, film from an MFC dispersion, comprising:
providing an MFC dispersion comprising a dispersing medium and a film forming component comprising about 50-100% by weight MFC,
applying a layer of the MFC dispersion to a support to form a wet MFC film,
subjecting the wet MFC film on the support to at least one drying step to form a dry MFC film,
measuring at least one parameter indicative of the dry solids content in the wet MFC film, or in the dry MFC film, or both at at least two data points which are laterally spaced across at least a width of the MFC film, and
varying drying conditions of said at least one drying step across a width of the wet MFC film in response to said at least one parameter.

2. The method as claimed in claim 1, wherein said measuring is performed after at least part of said at least one drying step.

3. The method as claimed in claim 1, further comprising:
at least one dewatering step prior to said at least one drying step, wherein said measuring is performed after at least part of said at least one dewatering step and before said at least one drying step.

4. (canceled)

5. The method as claimed in claim 1, wherein at least one of the at least two data points indicates a dry solids content at a position in a lateral edge portion of the MFC film.

6. The method as claimed in claim 1, wherein at least one of the at least two data points indicates a dry solids content at a portion that is laterally spaced from a lateral edge portion of the MFC film.

7. The method as claimed in claim 1, wherein said varying drying conditions comprises selectively varying a drying effect across a width direction.

8. The method as claimed in claim 7, wherein said varying a drying effect comprises reducing the drying effect at at least one lateral edge portion of the wet MFC film.

9. The method as claimed in claim 7, wherein said varying a drying effect comprises limiting an impingement width of a drying gas, or radiation, or both to be narrower than an MFC film width.

10. The method as claimed in claim 7, wherein said varying a drying effect comprises guiding drying gas away from at least one lateral edge portion of the MFC film.

11. The method as claimed in claim 7, wherein said varying a drying effect comprises extracting drying gas away from at least one lateral edge portion of the MFC film.

12. The method as claimed in claim 7, wherein said varying a drying effect comprises masking a lateral edge portion of the MFC film.

13. The method as claimed in claim 7, wherein said varying a drying effect comprises sealing, against a support surface laterally outside, a lateral edge portion of the MFC film.

14. The method as claimed in claim 7, wherein said varying a drying effect comprises selectively controlling a duty cycle, a combustion gas pressure, an intensity of a radiation source, or any combination thereof for providing said drying.

15. The method as claimed in claim 1, wherein said varying drying conditions comprises selectively injecting at least one of a cooling medium and a dispersing medium onto the wet MFC film

16. (canceled)

17. (canceled)

18. (canceled)

19. The method as claimed in claim 1, wherein the MFC dispersion has a dry solids content of about 2.5-25% by weight, and optionally a viscosity which is above about 4 Pas at a shear rate of 20 s⁻¹.

20. The method as claimed in claim 1, wherein an average film thickness of the dry MFC film is about 5-60 μm.

21. The method as claimed in claim 1, wherein a film weight of the dry MFC film is about 4-80 g/m².

22. (canceled)

23. (canceled)

24. The method as claimed in claim 1, wherein the film forming component comprises at least 60% by weight MFC.

25. The method as claimed in claim 1, wherein a film width of the dry MFC film is about 0.3-4 m.

26. The method as claimed in claim 1, further comprising:
measuring said at least one parameter indicative of the dry solids content in the wet MFC film, or in the dry MFC film, or both at at least two data points which are spaced from each other in a thickness direction of the MFC film.

27. A device for producing a microfibrillated cellulose, MFC, film from an MFC dispersion, comprising:
a support guiding device configured to guide a continuous support,
a casting device configured to apply an MFC dispersion as a wet MFC film on the continuous support,
a drying arrangement configured for removing a dispersing medium from the wet MFC film so as to form a dry MFC film,
at least one measuring arrangement configured for providing data indicative of a dry solids content in the wet MFC film, or in the dry MFC film, or both at at least two data points which are laterally spaced across at least a width of the MFC film, and
a controller, configured to receive said data, and to control the drying arrangement based on said data, so as to vary drying conditions across a width of the wet MFC film.

28. (canceled)

29. (canceled)

30. (canceled)

31. (canceled)

32. (canceled)

33. A microfibrillated cellulose, MFC, film having a longitudinal direction, which is parallel with a production direction of the MFC film and a width direction, which is perpendicular to the longitudinal direction,
the MFC film having a film forming component content of at least 80-99.9% by weight,
the film forming component comprising at least 50% by weight MFC,
the MFC film having a width of about 0.3-4 m,
the MFC film having a dispersing medium content of about 0.1-20% by weight, and
the dispersing medium content of the MFC film having a standard deviation which is less than 1% by weight along the width direction,
wherein a moisture content is analyzed for each cm of MFC film width.

34. (canceled)

35. (canceled)

36. (canceled)
